(19) United States
(12) Patent Application Publication
(10) Pub. No.: US 2024/0294996 A1
(43) Pub. Date: Sep. 5, 2024

(54) DEVICES THAT INCLUDE A DRIED REAGENT:SUBSTRATE COMPLEX AND METHODS FOR GENERATING SUCH COMPLEXES AND DEVICES

(71) Applicant: Argonaut Manufacturing Services, Inc., Carlsbad, CA (US)

(72) Inventor: Mark NOWAKOWSKI, San Diego, CA (US)

(73) Assignee: Argonaut Manufacturing Services, Inc., Carlsbad, CA (US)

(21) Appl. No.: 18/358,885

(22) Filed: Jul. 25, 2023

Related U.S. Application Data
(63) Continuation of application No. 18/177,085, filed on Mar. 1, 2023.

(51) Int. Cl. C12Q 1/70; B01L 3/00

(52) U.S. Cl. CPC C12Q 1/701; B01L 3/5023; B01L 3/502707; B01L 2200/16; B01L 2300/0654; B01L 2300/069; C12Q 1/6851

(57) ABSTRACT

The present invention provides a method for producing dried and stable reagent:substrate complexes suitable for use in various types of assays. Also provided are dried reagent:substrate complexes produced by the method of the invention, as well as methods for their use to perform an assay.

### RELATED APPLICATION(S)

This application is a continuation to commonly-owned U.S. patent application Ser. No. 18/177,085, filed 1 Mar. 2023, and claims the benefit of and priority to provisional patent application No. 63/315,330, filed on 1 Mar. 2022, which application is hereby incorporated by reference in its entirety for any and all purposes.

### BACKGROUND OF THE INVENTION

### Field of the Invention

The invention relates generally to reagents, and more specifically to a method for generating a dried reagent, or a combination of dried reagents, on a solid phase substrate, as well as use of the reagent or solid phase substrate including the dried reagent to perform an assay.

### Background Information

Numerous methods and systems have been developed for conducting chemical, biochemical, and/or biological assays. These methods and systems are essential in a variety of applications including medical diagnostics, food and beverage testing, environmental monitoring, manufacturing quality control, drug discovery, drug delivery and basic scientific research.

It is desirable that assay methods and devices have one or more of the following characteristics: 1) high throughput: 2) high sensitivity: 3) high precision and/or accuracy: 4) low cost: 5) low consumption of reagents: 6) multiplexing capability: 7) stability of reagents; and 8) shippability at room temperature. It is also desirable in many applications that these types of performance benefits are achieved with assay formats that are easy to carry out, are amenable to automation, use stable dry reagents, are efficient and less costly to manufacture, and/or require little or no manipulation before use.

A variety of approaches have been developed that provide reagents for assays in dry, stable form. Despite the known methods and devices for conducting assays, there exists a need for improved production of dried reagents using automation to increase throughput of production, as well as consistency and accuracy of assays using such reagents, especially for use in assays that employ the use of microchannel architecture in the assay cartridge that are not able to accommodate the use of conventional forms of lyophilization, such as lyophilized beads, due to the small dimensions of the chambers and capillary paths.

### SUMMARY OF THE INVENTION

The object of the invention is to provide new, patentable articles, compositions, and devices for conducting biochemical and biological assays in formats that employ improved dried, preferably lyophilized, reagents. Such articles, compositions, and devices will increase production throughput, as well as improve the consistency and accuracy of assays that utilize such reagents, especially when such assays are conducted in assay cartridges that employ microchannel architecture.

Thus, one aspect of the invention concerns dried reagent:substrate complexes that include a dried reagent species (i.e., a dried reagent having a defined chemical composition) disposed on a reagent deposit zone of a hydrophilicity-enhanced surface of a solid phase substrate. The dried reagent is typically formed from a known or measured volume (or volume equivalent (e.g., mass) of desired liquid reagent (i.e., a liquid reagent having a defined chemical composition, which definition can be the result of an extraction, separation, fractionation, combination, and/or addition of one or more components, for example, chemicals, etc.) deposited or otherwise disposed on a reagent deposit zone (i.e., some or all) of a hydrophilicity-enhanced surface of a solid substrate to form a reagent:substrate complex. Such liquid reagent volumes advantageously range from about 5000 μl to less than about 0.000001 μl prior to drying or any range in between, with volumes of about 1000, 500, 250, 100, 50, 25, 20, 15, 10, 9, 8, 7, 6, 5, 4, 3, 2, 1, 0.1, 0.01, 0.001, 0.0001, 0.00001, or 0.000001 μl prior to drying being particularly advantegous. Preferred drying or dehydration processes include lyophilization. Preferred solid phase substrates include flexible membranes or meshes, which flexible membranes or meshes include thin films or thin film meshes. In some embodiments, the substrate is formed from a polymer, for example, a plastic. In some embodiments, the solid phase substrate is formed from a flexible membrane or mesh that is soluble. Drying a reagent:substrate complex forms a dried reagent:substrate complex. Dried reagent:substrate complexes formed from different liquid reagents, different volumes of the same liquid reagent, and/or different substrates will be understood to constitute different dried reagent:substrate complexes or different dried reagent:substrate complex species.

In many embodiments, a dried reagent:substrate complex will have an attachment layer disposed on some or all of a surface of the solid phase substrate that is opposite that of the reagent deposit zone. In some embodiments, the attachment layer includes an adhesive or other bonding element that allows the dried reagent:substrate complex to be attached to a particular desired location, for example, on a surface of a particular location (e.g., in a specific reservoir, channel, reaction zone, detection zone, or the like) of a microfluidic molecular assay device.

In some embodiments, a dried reagent:substrate complex includes a dried reagent having a convex, concave, or flat upper surface and a substantially flat or smooth bottom surface. The upper surface of the dried agent is preferably exposed to the environment, which can facilitate reconstitution of the dried reagent upon exposure to a sufficient volume of a desired solvent or solution (e.g., water, a buffer, a liquid biological sample or a processed derivative or fraction thereof, etc.). The shape of the dried reagent's upper surface can be defined, at least in part, by the hydrophilicity-enhanced surface of the solid phase substrate upon which the liquid reagent was deposited prior to drying. Other factors that can influence the shape of the dried reagent's upper surface include the drying conditions employed to dry the reagent, whether the reagent deposit zone is bounded by or includes one or more structures, for example, a perimeter wall, one or more posts, etc. present on its surface. As will be appreciated, such additional structures can be included during the manufacture of the solid phase substrate. The dried reagent's substantially flat bottom surface is typically defined by the top or upper (or other) surface of the reagent deposit zone. In some embodiments, the surface of the reagent deposit zone upon which a liquid reagent is to be deposited can be flat, concave, convex, or otherwise patterned as desired. In some embodiments, such patterning can include one or more structures such as posts, ridges, channels, and the like.

In some embodiments, a dried reagent:substrate complex can be substantially disc-shaped when viewed from above, for example. In other embodiments, a dried reagent:substrate complex can be engineered to take any desired two-dimensional shape on the hydrophilicity-enhanced surface of a solid phase substrate. Such engineering includes, for example, providing one or more surface features onto the surface of the reagent deposit zone that interfaces with or contacts the liquid reagent when the liquid reagent is dispensed or deposited thereon. Representative two-dimensional shapes (e.g., when viewed from above) include those corresponding to polygons, e.g., triangles, rectangles, pentagons, hexagons, septagons, octagons, nonagons, decagons, crescents, ovals, ellipses, and combinations of any desired shape, particularly those that may be adapted for particular microfluidic assay devices or other architectures.

In many embodiments, the dried reagent component of a dried reagent:substrate complex according to the invention has a height that is reduced as compared to what the height of the dried reagent component would have been if the surface of the solid phase substrate was not a hydrophilicity-enhanced surface. In the context of the invention, a “dried reagent component” refers to a one or more dried reagent species disposed on the substrate.

In some embodiments, the dried reagent component of a dried reagent:substrate complex will comprise a plurality (i.e., two or more) of different dried reagent species, which different dried reagent species can be advantageously layered one on top of another. In other embodiments, the dried reagent component of a dried reagent:substrate complex may comprise two or more layers of the same dried reagent species. Yet other embodiments with three or more layers, the dried reagent component can contain at least two layers of the same dried reagent species. In some embodiments, the dried reagent component of a dried reagent:substrate complex comprises at least 2, 3, 4, 5, 6, 7, 8, or more substantially homogeneous layers each comprised of a different reagent formulation.

When the dried reagent component includes two or more dried reagent species (preferably layered one on top of another), each reagent layer is preferably being separated from the other reagent layer(s) by an intervening reagent separation layer. When multiple reagent separation layers are present in a dried reagent:substrate complex, each separation layer can be comprised of the same or different material or chemical composition.

In some embodiments, a dried reagent:substrate complex of the invention can be defined by a maximum height and/or a periphery dimension, for example, an approximated or substantially known length of an outer diameter or circumference. Advantageously, the maximum height of the dried reagent component of the complex is equal to or less than about 90, 80, 70, 60, 50, 40, 30, 20, or 10% of a periphery dimension such as the circumference dried reagent component of the dried reagent:substrate complex.

In many embodiments, a dried reagent:substrate complex, advantageously the dried reagent component thereof, will comprise a moisture content of less than about 10, 9, 8, 7, 6, 5, 4, 3, 2, or 1% by weight after drying, for example, by lyophilization.

In many embodiments, at least the reagent deposit zone(s) of the hydrophilicity-enhanced surface of the solid phase substrate is(are) treated to stabilize the dried reagent, wherein the treatment optionally is performed before or after deposition of a desired volume of a particular liquid reagent formulation.

In some embodiments, the reagent deposit zones on the solid phase substrate have defined positions in order to facilitate automated manufacture of large numbers of dried reagent:substrate complexes, e.g., 10-1,000,000 or more dried reagent:substrate complexes. As those in the art will appreciate, the number of dried reagent:substrate complexes produced will depend on such factors as whether the dried reagent:substrate complexes are produced in a continuous or batch manner, for example, by depositing a known volume of a particular desired liquid reagent at particular, preferably defined, locations (e.g., addresses defined by Cartesian (x and y) coordinates) on the hydrophilicity-enhanced surface of the solid phase substrate. In some embodiments, deposition of a known volume of the particular desired liquid reagent on the solid phase substrate defines the reagent deposit zone, while in other embodiments the location of one or more particular reagent deposit zones on the solid phase substrate is defined prior to liquid reagent deposition, i.e., at an “address” on the hydrophilicity-enhanced surface of the solid phase substrate. In some embodiments, aliquots of different liquid reagent species can be concurrently (i.e., at the substantially the same time) dispensed onto different reagent deposit zones on the same solid phase substrate. Aliquots of the same or different liquid reagent species can be dispensed in series or in parallel (i.e., two or more aliquots being concurrently dispensed onto different reagent deposit zones or layered on top of a liquid reagent species that had previously been deposited on the reagent deposit zone located on the same or a different solid phase substrate to form a dried reagent:substrate complex having two or more layers of different reagent species, which reagent layers may be separated by intervening reagent separation layers).

In many embodiments, the reagent deposit zone has a periphery that is bounded by a perimeter region of the solid phase substrate wherein the perimeter region comprises one or more recessed portions that extend partially or entirely through the substrate so that the dried reagent:substrate complex can be detached or removed from the rest of the substrate. In some embodiments, the perimeter region has at least about 1, 2, 3, 4, 5, or more recessed portions that extend around at least about 50, 60, 70, 80, 90, 95%, or more, even completely (i.e., 100%), about the perimeter region.

Another aspect of the invention concerns assay devices that include at least one dried reagent:substrate complex according to the invention. In many embodiments, such an assay device is a microfluidic device, advantageously a lateral flow diagnostic device.

In one aspect, the present invention provides a method of generating a reagent:substrate complex on a solid phase substrate. The method includes: a) depositing a liquid aliquot of a reagent on a solid phase substrate having a surface for contacting the reagent, wherein the surface is treated to increase the hydrophilicity of the surface; and b) treating the surface under conditions to partially or entirely desiccate the aliquot, thereby generating the dried reagent:substrate complex on the solid phase substrate. In some embodiments, the formed reagent:substrate complex has a disc shape including a convex or concave top surface.

In another aspect, the present invention provides a method of generating a reagent:substrate complex having a disc shape on a solid phase substrate. The method includes: a) depositing a liquid aliquot of a reagent on a solid phase substrate having a surface for contacting the reagent, wherein the surface is treated to increase the hydrophilicity of the surface; and b) treating the surface under conditions to partially or entirely desiccate the aliquot and form a solid phase disc, thereby generating the dried reagent:substrate complex on the solid phase substrate. In some embodiments, the formed reagent:substrate complex has a convex or concave top surface.

In another aspect, the invention provides a method of generating a hybrid reagent:substrate complex having at least two different reagents on a solid phase substrate. The method includes: a) depositing a first liquid aliquot of a first reagent on a solid phase substrate having a surface for contacting the reagent, wherein the surface is treated to increase the hydrophilicity of the surface: b) freezing the first aliquot forming a first homogeneous frozen layer of the first reagent: c) depositing a second liquid aliquot of a second reagent on the first homogeneous frozen layer: d) freezing the second aliquot forming a laminate having the first homogeneous frozen layer and a second homogeneous frozen layer of the second reagent; and e) treating the surface under conditions to lyophilize the laminate, thereby generating the hybrid reagent:substrate complex on the solid phase substrate.

In yet another aspect, the invention provides a reagent:substrate complex or a hybrid reagent:substrate complex produced by the method of the invention.

In still another aspect, the invention provides a solid phase substrate having a reagent:substrate complex or hybrid reagent:substrate complex of the invention formed on the surface of the solid phase substrate.

In another aspect, the invention provides an assay device for conducting a biological or chemical assay which includes a reagent:substrate complex, a hybrid reagent:substrate complex, or a solid phase substrate of the invention.

In another aspect, the invention provides a method of performing an assay utilizing a reagent:substrate complex, a hybrid reagent:substrate complex, an assay device or a solid phase substrate of the invention. The method includes contacting the dried reagent:substrate complex or hybrid reagent:substrate complex, whether alone or disposed in an assay device or on a solid phase substrate, with a test sample to produce a reaction mixture, and detecting an analyte in the reaction mixture.

### BRIEF DESCRIPTION OF THE FIGURES

In the drawings, like elements are assigned like reference numerals. The drawings are not necessarily to scale, with the emphasis instead placed upon the principles of the present invention. Additionally, each of the embodiments depicted are but one of a number of possible arrangements utilizing the fundamental concepts of the present invention. The drawings are briefly described as follows.

FIG. 1 is a top view of a solid phase substrate having a plurality of reagent deposit zones in one embodiment of the invention.

FIG. 2 is a partial exploded view of a solid phase substrate having reagent:substrate complexes formed on reagent deposit zones in one embodiment of the invention.

FIG. 3 is a top view of an assay device including a solid phase substrate having a reagent:substrate complex formed on its surface in one embodiment of the invention.

FIG. 4 is an elevated side view of the assay device shown in FIG. 3.

FIG. 5 illustrates a lateral flow device including at least one reagent:substrate complex manufactured by the method of the invention in one embodiment of the invention.

FIGS. 6A-6B show two photographs.

FIG. 6A is a top view image of four LyoDots each having a diameter of 3.75 mm.

FIG. 6B is a side view image of a LyoDot with a thickness of 390 μm. The LyoDots shown in this Figure were formed from the reagents needed for the CDC 2019-nCoV RT-PCR assay (Fortis Life Sciences).

FIGS. 7A-7F show six panels representing RT-qPCR results from experiments described in Example 2, below, which involved a CDC 2019-nCoV RT-PCR assay using LyoDot material (hatched lines in FIGS. 7D-7F) compared to the non-lyophilized liquid reagents (solid lines in FIGS. 7D-7F). In these experiments, synthetic SARS-CoV-2 RNA copy number was varied, while the concentration of RNase P template was held constant at Ing per reaction. Each sample was run in triplicate.

FIG. 7A is a bar chart of the Ct values for the CDC 2019-nCoV RT-PCR N1 assay at different copy numbers.

FIG. 7B is a bar chart of the Ct values for the CDC 2019-nCoV RT-PCR N2 assay at different copy numbers.

FIG. 7C is a bar chart of the Ct values for the RNase P template at a constant copy number with variable amounts of SARS-CoV-2 RNA.

FIG. 7D is an amplification curve for the CDC 2019-nCoV RT-PCR Nlassay.

FIG. 7E is an amplification curve for the CDC 2019-nCoV RT-PCR assay.

FIG. 7F is an amplification curve for the CDC 2019-nCoV RT-PCR RNase P assay.

FIG. 8 shows tabular data of the RT-qPCR results corresponding to the results from the experiments depicted in FIGS. 7A-7F for the CDC 2019-nCoV RT-PCR assay for LyoDot material compared to the non-lyophilized liquid reagents. Each condition was run in triplicate and contained SARS-CoV-2 RNA and RNase P template as indicated.

FIGS. 9A-9B show two photographs.

FIG. 9A depicts a device described in Example 2, below, that contains colored LyoDots in a chamber before addition of liquid (labeled “B” in the drawings), with the different colors being designated with “+” and “−” symbols.

FIG. 9B depicts the same device as FIG. 9A after the addition of water to the device. The LyoDots were attached to the chamber, which was made of acrylic and had dimensions of 0.5×0.5×0.030 inches. When exposed to liquid, the LyoDots readily dissolved and the differentially colored solutions (designated with “+” and “−” symbols) remained in their respective side of the chamber.

FIGS. 10A-10B contain two photographs.

FIG. 10A shows LyoDots (see Example 2, below) being handled by air tweezers.

FIG. 10B shows LyoDots presented on a sheet to air tweezers.

FIG. 11 is a photograph that shows LyoDots (see Example 2, below) configured in strips to illustrate a representative example of a lyophilized reagent configuration compatible with manufacturing automation.

### DETAILED DESCRIPTION OF THE INVENTION

The present invention is based on an innovative method for forming a dried reagent:substrate complex on a solid phase substrate in an automated, scalable workflow. The method allows for formation of one or more dried reagent:substrate complexes on the surface of the solid phase substrate where the reagent is deposited on a reagent deposit zone on the surface in a small volume of fluid and treated under conditions that result in desiccation of the liquid reagent and formation of a dried solid reagent. Treatment of the surface of the solid phase substrate to increase hydrophilicity allows formation of a dried reagent having a reduced height dimension as opposed to a spherical bead shape having a uniform diameter. This is important as many in-vitro diagnostic applications utilize cartridges with microfluidic dimensions which cannot accommodate lyophilized beads with a diameter greater than 1 mm. The method of the invention allows for generation of solid, dried reagent:substrate complexes that have a reduced height dimension making them suitable for use in applications that cannot accommodate reagent beads. Additionally, treating the surface of the solid phase substrate to increase hydrophilicity of the surface before a liquid reagent is deposited on the surface vastly decreases the time required to treat the liquid reagent to desiccate the liquid and transform it to a dried and stable solid, as well as allowing generation of dried reagent:substrate complexes that include homogeneous layers of different reagents in a single reagent deposit zone.

Those familiar with the art of modification of polymer surfaces which form the solid phase substrate by such means as plasma treatment, will appreciate that varying degrees of surface modification will results in varying degrees of surface energy and consequently varying degrees of hydrophilicity. In one embodiment of the invention, if the solid phase substrate is not modified in any way, the dried reagent:substrate complex after treatment, such as lyophilization, can easily be removed from the solid phase substrate. In another embodiment, if the solid phase substrate is modified and rendered hydrophilic, the dried reagent:substrate complex after lyophilization becomes anchored to the solid phase substrate and cannot easily be removed from the reagent deposit zone of the solid phase substrate. It is important to note that the solid phase substrate thickness is very small, allowing the lyophilized dried reagent:substrate complex anchored to the film (or mesh) to be easily accommodated in an assay device, such as a microchannel assay device. In addition, the nature of anchoring the lyophilized dried reagent:substrate complex to the reagent deposit zone of the solid phase substrate results in a dried reagent component that is much easier to handle in an automated assembly process due to the robust nature of the two-piece component (i.e., the dried reagent and substrate components.

In various embodiments, a dried reagent:substrate complex or substrate including a dried reagent:substrate complex produced by the method of the invention requires no further manipulation before use to perform an assay, is less costly and more efficient to produce, and results in a simplified user workflow. As described herein, a dried reagent:substrate complex produced by the method of the invention may be incorporated into a stand-alone assay device, such as a lateral flow device, or used directly by the end user to conduct a single type of assay, or multiple types of assays, without the need to add additional reagents, thereby increasing reliability of a respective assay result and reducing inconsistencies due to lot-to-lot variation in reagents.

Before the present articles, compositions, and methods are described, it is to be understood that this invention is not limited to the particular articles, compositions, methods, and experimental conditions described, as such articles, compositions, methods, and conditions may vary. It is also to be understood that the terminology used herein is for purposes of describing particular representative embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this invention and so forth. As used herein, the term “about”, when used in conjunction with a numerical value, refers to approximately a +/−10% variation from the stated value.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

A “patentable” composition, process, machine, or article of manufacture according to the invention means that the subject matter at issue satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically excludes the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances

The present invention provides among its various aspects patentable methods of generating a dried reagent:substrate complex on a solid phase substrate. The dried reagent:substrate complex or substrate having a dried reagent:substrate complex may be used in a variety of applications, such as biological and chemical assays, as well as in-vitro diagnostics and therapeutics.

Accordingly, in one aspect, the present invention provides a method of generating a dried reagent:substrate complex on a solid phase substrate, advantageously on a reagent deposit zone on a hydrophilicity-enhanced surface of the solid phase substrate. The method includes: a) depositing a liquid aliquot of a desired reagent on a solid phase substrate having a surface for contacting the reagent, wherein the surface has been treated to increase the hydrophilicity of the surface; and b) treating the liquid reagent on the surface of the reagent deposit zone under conditions to at least partially desiccate the aliquot and form a solid, wherein the increased hydrophilicity of the surface causes the liquid reagent to form a shape having a reduced height dimension as compared to the height that would have resulted from the process had the surface not been treated to enhance its hydrophilicity, thereby generating the dried reagent:substrate complex on the solid phase substrate.

In various embodiments, a liquid aliquot of a reagent deposited on the surface of the substrate is treated under conditions to desiccate the aliquot and form a stable dried solid. In various embodiments, treating includes one or more of lyophilization, increased temperature, or decreased pressure. However, it will be understood that any conventional method known to reduce the solvent content of a liquid may be utilized. In various embodiments, desiccation produces a stable dried reagent:substrate complex that has a residual moisture of less than about 10, 9, 8, 7, 6, 5, 4, 3, 2 or 1%.

As used herein, “lyophilization” is the removal of solvent from the frozen state by sublimation. Lyophilization is accomplished by freezing the solution below its melting point and then manipulating the temperature and pressure to provide sublimation. Precise control of temperature and pressure permits drying from the frozen state without product melt-back. In practical applications, the process is accelerated and more precisely controlled under reduced pressure conditions.

As used herein, “lyophilizate” is the solid, powder or granular material remaining after lyophilization. The solid, powder or granular material is essentially free of solvent.

In various embodiments, desiccation is achieved by lyophilization. In some embodiments the reagents deposited on the surface are treated under lyophilization conditions for less than about 25, 24, 23, 22, 21, 20, 19, 18, 17, 16, 15, 14, 13, 12, 11, 10, 9, 8, 7, 6, 5, 4, 3, 2, or 1 hour to form a dried reagent:substrate complex. In some embodiments, the lyophilization time required to form the dried reagent:substrate complex is reduced as compared to the lyophilization time required to form the dried reagent:substrate complex on an untreated surface. For example, in some embodiments the lyophilization time is reduced by greater than about 10, 20, 30, 40, 50, 60, 70, 80, 85, 90 or 95% as compared to the lyophilization time required to form the dried reagent:substrate complex on an untreated surface.

In various embodiments, the solid phase substrate includes a surface for receiving a liquid reagent that is treated to increase the amount of hydrophilicity of the surface. The intended deposition locations for liquid reagent aliquots on the surface of the solid phase substrate are referred to as “reagent deposit zones”. Increasing the hydrophilicity of the substrate's surface, particularly in the regions intended as reagent deposit zones, allows a liquid droplet deposited on the surface to spread across the surface and produce a shape having a reduced height, such as a disc, as compared to a more hemispherically shaped liquid droplet that would form had the surface not been treated to enhance or increase its hydrophilicity. Any suitable surface treatment or surface modification technique known to enhance, increase or other improve the hydrophilicity of a some or all of the surface of a solid phase substrate may be used to alter the hydrophilicity of the surface. Examples of such techniques include, but are not limited to, oxygen plasma treatments to render hydrophobic material surfaces more hydrophilic, the use of wet or dry etching techniques to smooth (or roughen) silicon surfaces, adsorption and/or grafting of polyethylene oxide or other polymer layers to substrate surfaces to render them more hydrophilic and less prone to non-specific adsorption of biomolecules and cells, the use of silane reactions to graft chemically-reactive functional groups to otherwise inert silicon surfaces.

With regard to enhancing the hydrophilicity of a surface, the surface may be treated or coated to reduce the contact angle of a liquid reagent on the surface to about 90 degrees or less. In many embodiments, the hydrophilicity of a surface is increased such that the contact angle of the surface is less than about 85, 80, 75, 70, 65, 60, 55, 50, 45, 40, 35, 30, 25, 20, 15, or 10 degrees.

In addition to treating a surface to increase hydrophilicity, the surface may be functionalized. A surface may be referred to as “functionalized” when it includes a linker, a scaffold, a building block, or other reactive moiety attached thereto, whereas a surface may be “nonfunctionalized” when it lacks such a reactive moiety attached thereto.

A functionalized surface may refer to the surface of the solid phase substrate comprising a functional group. A functional group may be a group capable of forming an attachment with another functional group. For example, a functional group may be biotin, which may form an attachment with streptavidin, another functional group. Exemplary functional groups may include, but are not limited to, aldehydes, ketones, carboxy groups, amino groups, biotin, streptavidin, nucleic acids, small molecules (e.g., for click chemistry), homo- and hetero-bifunctional reagents (e.g., N-succinimidyl(4-iodoacetyl) aminobenzoate (STAB), dimaleimide, dithio-bis-nitrobenzoic acid (DTNB), N-succinimidyl-S-acetyl-thioacetate (SATA), N-succinimidyl-3-(2-pyridyldithio) propionate (SPDP), succinimidyl 4-(N-mafeimidomethyl)-cyclohexane-1-carboxylate (SMCC) and 6-hydrazinonicotimide (HYNIC), and antibodies. In some instances, the functional group is a carboxy group (e.g., COOH). Additionally, photodeprotection techniques can be used to selectively activate chemically-reactive functional groups at specific locations of the surface, for example, the selective addition or activation of chemically-reactive functional groups such as primary amines or carboxyl groups on the surface may be used to covalently couple oligonucleotide probes, peptides, proteins, or other biomolecules to the surface. In general, the choice of surface treatment or surface modification utilized will depend both on the type of surface property that is desired and on the type of material from which the substrate is made.

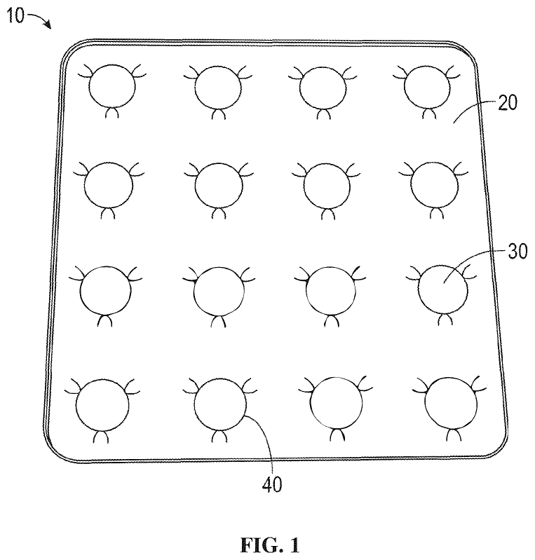

FIG. 1 shows a thin planar solid phase substrate utilized in the method of the invention in one embodiment. The solid phase substrate 10 includes a top surface 20 which is treated to increase the hydrophilicity of the surface and make it more suitable for receiving an aliquot of a liquid reagent. In some embodiments, as shown in FIG. 1, the substrate 10 includes one or more reagent deposit zones 30 upon which the liquid reagent is deposited. Each reagent deposit zone 30 is defined by a perimeter that includes one or more recessed regions 40 extending partially or entirely through the substrate 10 such that the reagent deposit zone 30 having a formed dried reagent:substrate complex thereon can be easily detached from the substrate 10.

As will be appreciated, the recessed regions 40 may be formed in the substrate by a variety of techniques known in the art. For example, the recessed regions may be formed by laser cutting, die cutting, etching and the like. In the embodiment shown in FIG. 1, each reagent deposit zone 30 is circular and has three recessed regions extending through the substrate 10 and is attached to the substrate 10 at three attachment points that can easily be broken by manual manipulation to separate a formed dried reagent:substrate complex from the substrate 10 for use. However, it will be understood that the reagent deposit zone 30 may have any shape perimeter, e.g., square, triangular, elliptical, and the like, and include any number of recessed regions to facilitate detachment of the reagent deposit zone from the substrate 10.

As discussed herein, the method includes depositing a liquid aliquot of a reagent on the surface 20 of the solid phase substrate 10. In various embodiments, the method utilizes small volumes of liquid reagent thereby reducing cost of manufacture. In embodiments the amount of liquid reagent deposited in each aliquot is equal to or less than about 5000, 1000, 500, 250, 100, 50, 25, 20, 15, 10, 9, 8, 7, 6, 5, 4, 3, 2, 1, 0.1, 0.01, 0.001, 0.0001, 0.00001, or 0.000001 μl.

In various embodiments, the substrate 10 may be composed of a variety of materials and may be flexible, semi-flexible, or rigid. Suitable materials can include, glass, ceramics, metals, plastics, polymers and combinations thereof. The material can be a solid sheet, film, or mesh. The materials for construction of the substrate include non-polymeric and polymeric materials. In many embodiments, one or more of the following, non-limiting examples of materials may be used in construction of the substrate, including plastics (e.g., cyclic olefin copolymer (COC), cyclic olefin polymer (COP), polyethylene terephthalate (PET), polyethylene (PE), high-density polyethylene (HDPE), low-density polyethylene (LDPE), polyvinyl chloride (PVC), polypropylene (PP), polystyrene (PS), high impact polystyrene (HIPS), polyamides (PA), acrylonitrile butadiene styrene (ABS), polyethylene/acrylonitrile butadiene styrene (PE/ABS), polycarbonate (PC), polycarbonate/acrylonitrile butadiene styrene (PC/ABS), polymethylmethacrylate (PMMA)), metals, elastomers (e.g., polydimethylsiloxane (PDMS)), glass (e.g., borosilicate), ceramics, and composite materials, such as carbon fiber composites.

As described, the substrate can be a solid, film, or mesh (i.e., a material comprised of a network of fibers, threads, or wires). In some embodiments that utilize solid or film substrates, the substrate is machined, processed, or otherwise treated to introduce holes or pores in the substrate. Such holes or pores can be of any desired shape, for example, substantially cylindrical, and can be evenly or randomly spaced. Holes or pores can be introduced by any suitable method, including punching, stamping, machining, or laser cutting. Processing to introduce holes or pores can be introduced before or after treatment to enhance surface hydrophilicity. Preferred hole or pore size ranges from about 0.001 mm to about 5 mm (or any range within this range), with holes or pores having a diameter ranging from about 0. 01 mm to about 4 mm being particularly advantageous. Examples or particular hole or pore diameters include about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, and 4.0 mm, as well as any diameter size or size range between any of these values. In the embodiments that employ mesh substrates, the mesh can be formed from any suitable material (or combination of different materials), for example, metals, plastics, fibers, or other flexible or ductile materials. As with solid substrates that have holes or pores, the openings in meshes suitable for use in context of the invention can be of any suitable size, including from about 0.001 mm to about 5 mm (or any range within this range), with opening sizes ranging from about 0. 01 mm to about 4 mm being particularly advantageous. Examples or particular opening sizes include about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, and 4.0 mm, as well as any opening size or size range between any of these values. As will be appreciated, holes, pores, openings in mesh, and the like can also help to secure a dried reagent component to the reagent deposit zone of the substrate.

In various embodiments, the substrate 10, or portions thereof may be formed from a material which is compatible with biological material and/or molecules (e.g., biocompatible), such as cells or cellular components (e.g., nuclei, perinuclear compartments, nuclear membranes, mitochondria, chloroplasts, or cell membranes), proteins (e.g., antibodies, or membrane, trans-membrane or cytosolic proteins), oligonucleotides, lipids, polysaccharides, nucleic acids, viral particles, ribosomes, hormones, ions or cofactors.

Once a liquid reagent aliquot is deposited on the surface 20 of a desired substrate, for example, on a reagent deposit zone 30, the surface is treated under conditions to desiccate, at least partially or entirely, the aliquot and form a stable dried reagent:substrate complex.

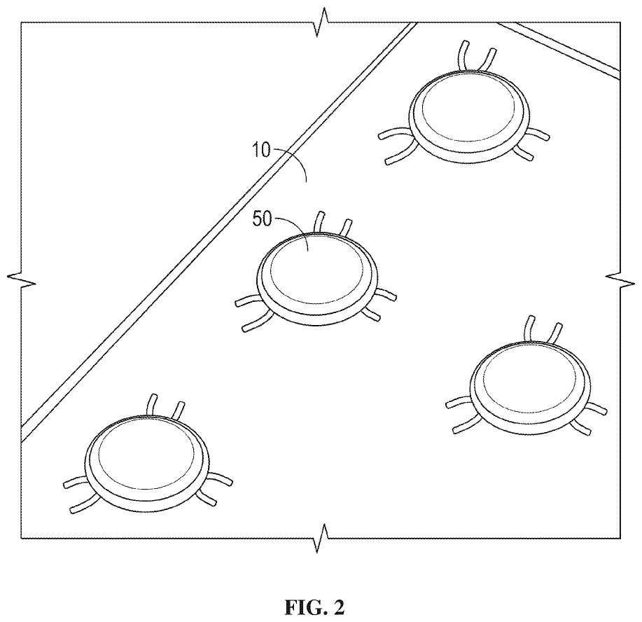

FIG. 2 shows a substrate 10 having formed dried reagent:substrate complexes 50 thereon. In various embodiments, once the surface of the substrate has been treated to form the dried reagent:substrate complex, the surface, including the dried reagent:substrate complex, may be covered with a substrate layer to package the substrate and dried reagent:substrate complex. For example, a flexible membrane, such as a thin polymeric film, may be utilized to cover the surface.

In addition to producing a dried reagent:substrate complex having a single type of reagent, the methodology of the invention provides for generating a dried hybrid reagent:substrate complex that includes at least two different types of reagents. To prevent mixing and/or reaction of liquid reagents during production of dried hybrid reagent:substrate complexes, one or more successive freeze cycles is performed as one type of liquid reagent is deposited on a different type of reagent. The resulting dried hybrid reagent:substrate complex includes distinct homogeneous layers of different reagents.

As such, the invention provides a method of generating a dried hybrid reagent:substrate complex having at least two different reagents on a solid phase substrate. The method includes: a) depositing a first liquid aliquot of a first reagent on a solid phase substrate having a surface for contacting the reagent, wherein the surface is treated to increase the hydrophilicity of the surface: b) freezing the first aliquot forming a first homogeneous frozen layer of the first reagent: c) depositing a second liquid aliquot of a second reagent on the first homogeneous frozen layer: d) freezing the second aliquot to form a reagent laminate having the first homogeneous frozen layer and a second homogeneous frozen layer of the second reagent; and e) treating the surface under conditions to lyophilize the laminate and form a solid phase, wherein the increased hydrophilicity causes the laminate to form a shape having a reduced height dimension, thereby generating the dried hybrid reagent:substrate complex on the solid phase substrate.

It will be appreciated that utilizing successive freeze cycles as reagents are deposited, can generate a stable and dried hybrid reagent:substrate complex having any number of different reagents disposed in homogeneous layers. In various embodiments, the dried hybrid reagent:substrate complex includes 2, 3, 4, 5, 6, 7, 8, 9, 10, or more homogeneous layers each having a different reagent.

The dried reagent:substrate complex or dried hybrid reagent:substrate complex produced by the method of the invention may include any reagent that can be dried from a liquid form and then reconstituted prior to use, for example in an assay. Examples include, but are not limited to, binding reagents useful in binding assays, enzymes, enzyme substrates, indicator dyes and other reactive compounds that may be used to detect an analyte of interest (i.e., target analyte). The assay reagents may also include substances that are not directly involved in the mechanism of detection but play an auxiliary role in an assay including, but not limited to, blocking agents, stabilizing agents, reducing agents, oxidizing agents, chaotropic agents, detergents, salts, pH buffers, preservatives, diluents and excipients. The reagents may also include biological cells or molecules including, but not limited to, cells, proteins (e.g., antibodies, and membrane, trans-membrane and cytosolic proteins), oligonucleotides, lipids, polysaccharides, nucleic acids, viral particles, ribosomes, antigens, hormones, ions and cofactors.

In some embodiments, the reagent includes a time release component that alters the solubility of the reagent. In this manner, a dried hybrid reagent:substrate complex may be produced in which different layers reconstitute at different times when utilized in an assay. In one embodiment, a dried hybrid reagent:substrate complex includes at least one homogenous reagent layer that includes a time release component, such as a water soluble polymer or hydrogel.

In embodiments where the solid phase substrate is flexible, it may be produced using a reel-to-reel manufacturing process. For example, the substrate may be formed by providing a stock reel for each layer type, e.g., solid phase substrate and optionally the coating substrate layer, and a final reel holding the formed substrate. The reels can be rotated to transfer material from the stock reels and deposit the formed substrate on the final reel. In embodiments, the solid phase substrate can pass through various stages as the substrate is passed between the reels. For example, the solid phase substrate or portions thereof can pass through a stage at which aliquots of liquid reagents are deposited on the substrate, advantageously on reagent deposit zones. The substrate may then be subjected to different treating procedures to form the dried reagent:substrate complexes on the surface of the substrate.

In various embodiments that solid phase substrate having one or more dried reagent:substrate complexes formed thereon may include a substrate layer disposed over the entire solid phase substrate, or over the dried reagent:substrate complexes to prevent contamination or degradation of the dried reagent:substrate complexes once formed. The substrate layer may be a thin film that is peelable from the solid phase substrate.

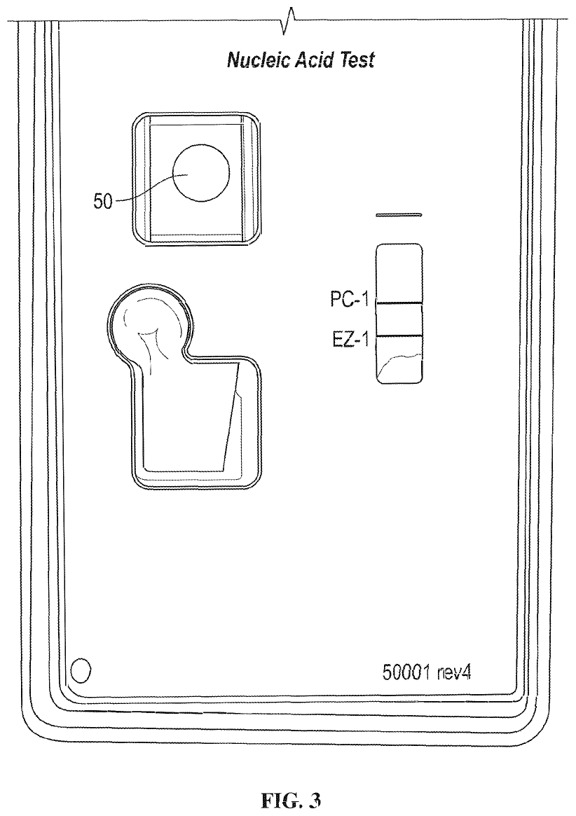

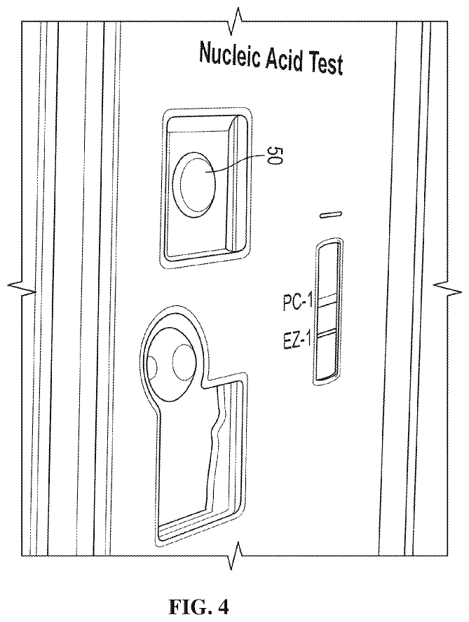

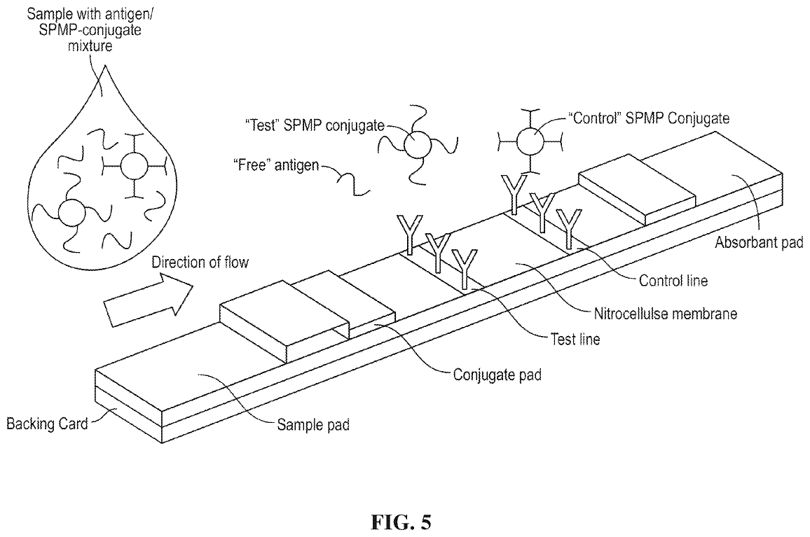

In embodiments, the solid phase substrate generated by the method of the invention includes dried reagent:substrate complexes of different types of reagents disposed on different location of the surface which are configured to perform an assay on the solid phase substrate. For example, the solid phase substrate may be incorporated into an assay device as shown in FIGS. 3-5, such as a lateral flow assay device as shown in FIG. 5.

A typical lateral flow device is illustrated in FIG. 5. Typical lateral flow devices include a sample pad for receiving a liquid sample, and additional regions/zones downstream of the sample pad which may include for example, a conjugate pad, one or more zones including reagents and a detection zone. In practice, a liquid sample is deposited on the sample pad and then flows downstream through the various regions. In one embodiment, the invention provides a lateral flow assay device which includes a solid phase substrate produced by the method of the invention having one or more dried reagent:substrate complexes thereon. In one embodiment, the dried reagent:substrate complex may be disposed integral with or adjacent the sample pad such that liquid sample deposited on the sample pad rehydrates the dried reagent:substrate complex. The reaction mixture of test sample and rehydrated reagent is then wicked downstream toward a detection zone and may involve rehydration of one or more additional dried reagent:substrate complexes as the reaction mixture flows downstream. While the invention is illustrated using a lateral flow device as shown in FIG. 5, it will be appreciated that the invention is not limited to this specific lateral flow device but rather may be used in any conventional lateral flow device that includes a sample receiving pad. Illustrative examples of lateral flow assay devices which may incorporate the device of the invention as a sample pad include, but are not limited to those disclosed in U.S. Pat. Nos. 10,073,091, 9,989,527, 9,709,562, 8,846,319, 9,944,922, 9,915,657, 8,822,151, 8,580,572, 8,153,444, 7,858,396, 7,910,381, 7,537,937, 7,344,893, 6,924,153, 6,372,513 and 6,656,744, each of which is incorporated herein by reference.

In various aspects, the invention provides a method of performing an assay. In one embodiment, the method utilizes the solid phase substrate of the invention configured for immersing a portion of the substrate into a liquid test sample or depositing a liquid test sample onto the surface of the substrate, e.g., a lateral flow assay device shown in FIG. 3.

In various embodiments, the test sample may be a liquid, gas or solid. Depending on the type of assay being performed and the type of sample being utilized, one in the art would appreciate that one or more excipients or diluents may be utilized with the test sample to facilitate rehydration of the dried reagent:substrate complex(s) and contact between the reagent(s) and the test sample.

Depending on the type of assay being conducted, a control system such as a computer or other automation device may be used to monitor and control the operation of the assay device of the invention, and/or to analyze obtained data. In embodiments, the solid phase substrate may include circuitry for controlling the assay and/or monitoring the assay and optionally be in operable connection to a control system. In certain embodiments, the circuitry is flexible so as to be used in a flexible substrate. In embodiments, the circuitry is operable to heat, cool or otherwise manipulate that reaction mixture within the chamber. For example, the circuitry may be operable to control a PCR reaction and/or a nucleic acid hybridization reaction.

The assay device of the invention may include one or more channels to facilitate use in, or with, an automated system, such as a biological or chemical analyzer. The one or more channels may be provided in the substrate and provide a fluid pathway between dried reagent:substrate complexes and the automated system or analyzer for passage of a reaction mixture. Such channels may be microchannels to facilitate use with a microfluidics system. For example, the solid phase substrate of the invention may include microchannels to allow fluid flow into and out of regions having dried reagent:substrate complexes for further analysis or manipulation of a reaction mixture by a microfluidics system.

The following examples are provided to further illustrate the advantages and features of the present invention, but it is not intended to limit the scope of the invention. While this example is typical of those that might be used, other procedures, methodologies, or techniques known to those skilled in the art may alternatively be used.

### Example I

### Formation of Dried Reagent:Substrate Complexes on a Solid Phase Substrate

A solid phase substrate having dried reagent:substrate complexes was generated using the methodology of the invention as follows.

1X excipient solution was formulated that would typically be used in the lyophilization of a PCR master mix.

A 0.015″ polycarbonate film was plasma treated to render the surface hydrophilic (a contact angle of less than 90 degrees). Note that if the film surface was not modified and a 4.7 μL drop of the excipient solution was applied to the surface, the solution would appear to be a spherical drop with a diameter of approximately 2.1 mm. However, when the surface is modified using plasma treatment, the same 4.7 μL drop appeared to be a slightly concave disc with a thickness of about 0.48 mm.

The polycarbonate film containing multiple liquid reagent dispenses was then placed on a precooled (−40 C) shelf and allowed to freeze for approximately 15 minutes.

The discs were then lyophilized for approximately 22 hours.

The films containing the lyophilized discs were harvested by simply inverting the film and collecting the lyophilized discs in bulk. In another embodiment the film could be modified such that the discs are retained on the surface of the film. Note that the lyophilized discs were approximately 4 mm in diameter with a thickness of approximately 0.48 mm.

The lyophilization procedure was optimized and repeated and dried reagent discs were formed using a lyophilization time of less than 1 or 2 hours.

### Example 2

### Lyodots™—Another Lyophilization Format Having Freeze-Dried Reagent:Substrate Complexes on a Solid Phase Substrate

### Prolog.

This Example 2 demonstrates a new configuration of lyophilized material where a lyophilized reagent is prepared in the form of a flat, circular, dome shape called a LyoDot™ (Argonaut Manufacturing Services, Carlsbad, CA). This reagent configuration accommodates small spaces and allows for rapid dissolution/resuspension of the lyophilized material, which are important criteria when designing and manufacturing a shallow-depth chambers are found in many microfluidic diagnostic devices.

To create a LyoDot, a liquid reagent is applied to a substrate, e.g., a film or mesh, having a surface treated to enhance its hydrophilicity and subsequently lyophilized. The substrate provides mechanical support, and the underside of the substrate allows, for example, adhesive application of one or more LyoDots to be attached to one or more specific locations in a microfluidic device, it being understood that the surface of LyoDot substrate opposite to that on which the liquid reagent was dispensed.

LyoDots can be easily integrated into device assemblies using any suitable approach, for example, standard “pick and place” automation due to their robust nature and planar X-Y coordinate (Cartesian) layout. As a proof of principle, and as described in this Example, LyoDots were created using RT-qPCR assay reagents from Fortis Life Sciences and multiplexed oligonucleotides corresponding to the CDC 2019 nCoV RT PCR assay. The LyoDots had a diameter of 3.75 mm and a thickness of 390 μm. When the LyoDots were functionally tested, assays resulting from their use were found to produce results that were comparable to results obtained using the liquid reagents prior to lyophilization.

To further highlight utilization of LyoDots in diagnostic device applications, this Example also describes the results obtained from using two LyoDots, each containing a different dye, after placing them in a chamber that was 0.5×0.5×0.030 inches. The LyoDots were placed adjacent to each other and were attached to the surface via adhesive, which was applied to the underside of a clear film substrate. Upon exposure to liquid, the LyoDots readily reconstituted and the resulting solutions remained on their respective side of the chamber. These dye-containing LyoDots exhibited attributes of fast dissolution and the ability to maintain spatial control of reconstituted lyophilized material, highlighting the attractiveness of using LyoDots in microfluidic devices or small point-of-care tests, which are rapidly growing segments in the molecular diagnostics market.

This Example, which demonstrates LyoDot functionality and implementation in a microfluidic device, shows the applicability of LyoDots to many types of microfluidic devices. Furthermore, this Example shows that LyoDots enable high volume, cost effective, “automation friendly” formats required for present and future generations of point-of-care testing equipment and methods.

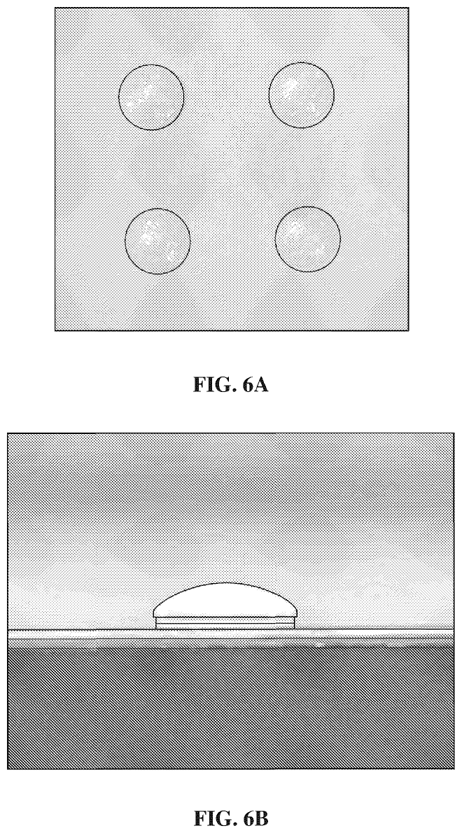

Introduction. The COVID-19 pandemic highlighted the need for rapid and sensitive diagnostic tests that are stable at room temperature and can be easily transported. One way to achieve this is through the incorporation of lyophilized reagents. A variety of lyophilization formats are known. For example, LyoDose™ bead technology (Argonaut Manufacturing Services, Carlsbad, CA), which consists of a sphere with lyophilized reagents in amounts needed for a single reaction in a device or well, has been utilized by many companies to enhance their products. Although the spherical shape of a LyoDose bead is compatible with many devices, the desire to reduce device footprint requires a thinner lyophilized material geometry. This Example describes a new configuration of lyophilized material where the lyophilized reagent is in the form of a circular, dome-shape called a LyoDot™ (FIGS. 6A-6B).

A significant advantage of the LyoDot configuration is that it allows a similar amount of lyophilized material to fit into thinner spaces as compared to conventional, bead-shaped lyophilized reagents (e.g., LyoDose beads) that contain comparable amounts of lyophilized material. For example, a LyoDot made from a 5 μL aliquot of liquid reagent (before lyophilization) was more than five times thinner than a spherical bead made with the same volume of liquid reagent. In general, a LyoDot is created by applying a liquid reagent to the surface of a substrate such as a thin film or mesh that has been treated to increase the hydrophilicity of the surface to which the liquid reagent is applied, followed by lyophilization. The substrate provides mechanical support, and the surface of the substrate opposite that to which the liquid reagent is applied can be used for adhesive application, which adhesive can then be used to attach the substrate to a specific location in a diagnostic device (e.g., a microfluidic device configured to perform a molecular diagnostic assay such as an immunoassay, a nucleic acid-based pathogen detection assay, etc. As will be appreciated, surface-modified substrates upon which liquid reagents are lyophilized, such as LyoDots, can be easily integrated into diagnostic devices during assembly using standard “pick and place” automation due to their robust nature and planar X-Y coordinate layouts. These attributes will further enhance device miniaturization while also lowering reagent and diagnostic device manufacturing cost.

### LyoDot Technology.

A representative example of LyoDot technology involved the preparation of material to perform a CDC 2019-nCoV RT-PCR assay. Here, LyoDots containing RT-qPCR master mix (Empirical Bioscience, Inc.) to perform an RT-qPCR assay (N1 assay=FAM, N2 assay=HEX, RNase P assay=ROX) combined with multiplexed oligonucleotides were prepared. These LyoDots had a diameter of 3.75 mm and a thickness of 390 μm (see FIGS. 6A-6B). Synthetic SARS-CoV-2 RNA template (Twist Bioscience) was added to the RT-qPCR reactions (20 μL final volume per reaction) at 5×10⁵, 5×10⁴, 5×10³, 5×10², 5×10¹, or 0 (negative control) copies. To simulate a clinical sample, Universal Human Reference RNA remained constant at 1 ng per reaction for each condition. LyoDots were resuspended in molecular grade water at 15 μL of water per one LyoDot, and the resulting suspension was distributed to qPCR plates at 15 μL volumes per well. Then, template (5 μL/reaction) was added to the wells, followed by RT-qPCR. Non-lyophilized liquid reagents were also tested. Samples were tested in triplicate for each condition.

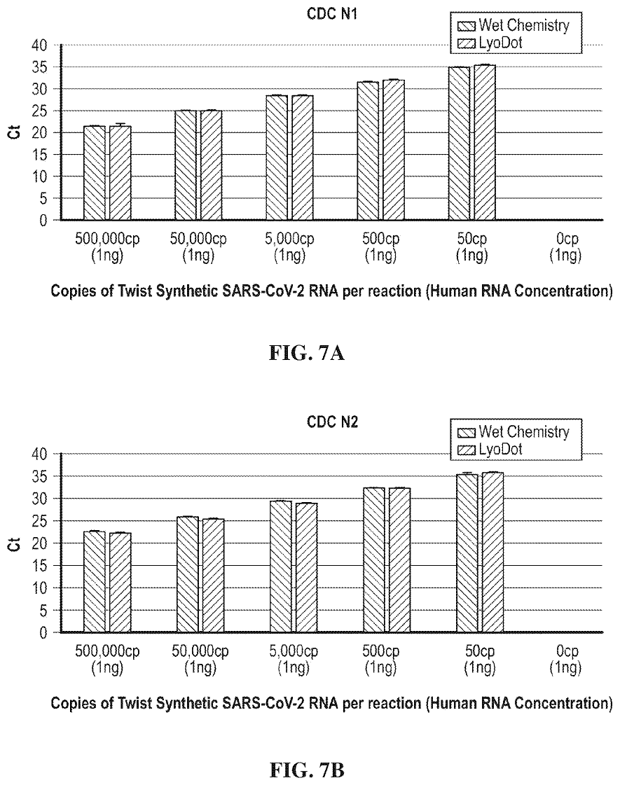

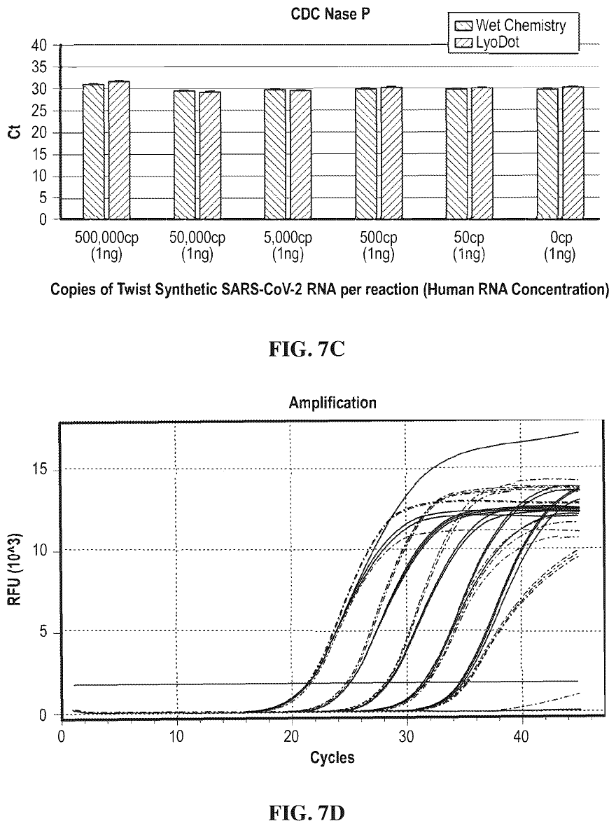

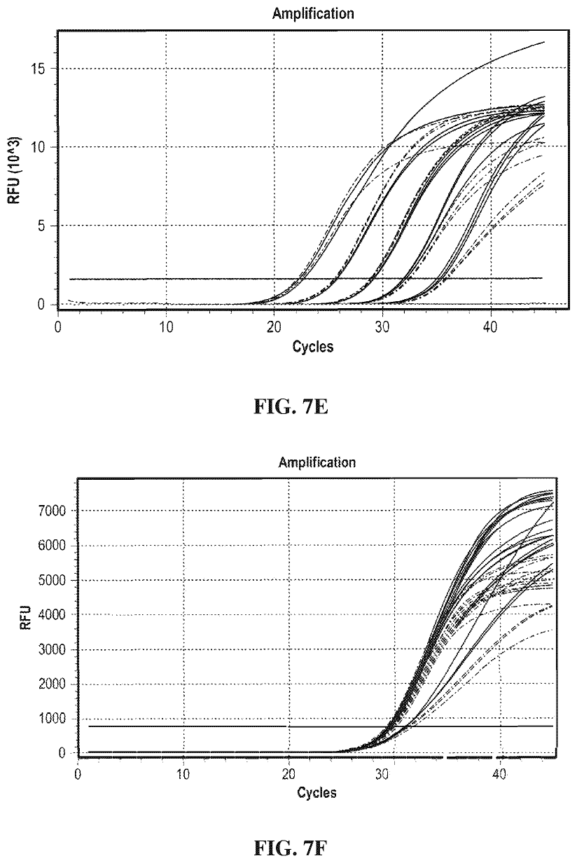

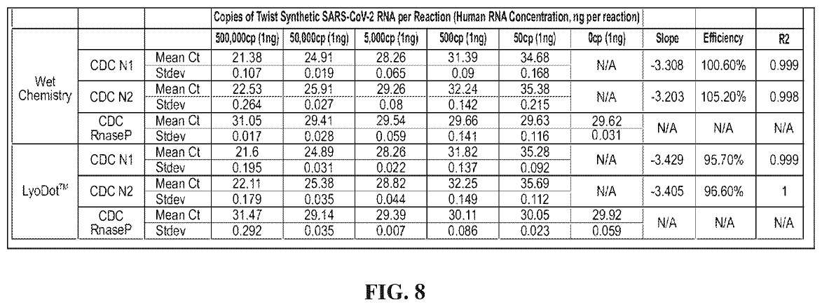

The results of this RT-qPCR testing are shown in FIGS. 7A-7F and FIG. 8. The resuspended LyoDot material demonstrated similar dynamic range and sensitivity when compared to the non-lyophilized liquid reagents. Negative controls (no nCoV template and with Universal Human Reference RNA) demonstrated amplification for RNase P while N1 and N2 had no detectable amplification. The CDC 2019-nCoV RT-PCR N1 and N2 assays displayed nearly 100% PCR reaction efficiency for both sets of reagents (lyophilized and liquid), with nearly identical Ct values. The CDC 2019-nCoV RT-PCR RNase P assay Ct values remained similar throughout the experiment, except when the copy number of nCoV synthetic RNA was at 500,000 copies per reaction, in which case RNase P was 2 Ct higher. This higher Ct was found in both non-lyophilized liquid reagents and LyoDots and was likely due to reagent component (e.g., oligonucleotides, dNTPs) depletion during amplifying the high copy RNA (here, the N1 and N2 targets), which led to less efficient amplification of the lower copy RNase P target. Overall, these results demonstrate equivalent performance of biochemical assays (here, RT-qPCR assays) using LyoDots and the non-lyophilized liquid reagents.

### Example 3

### Use of Lyodots™ in a Microfluidic Device

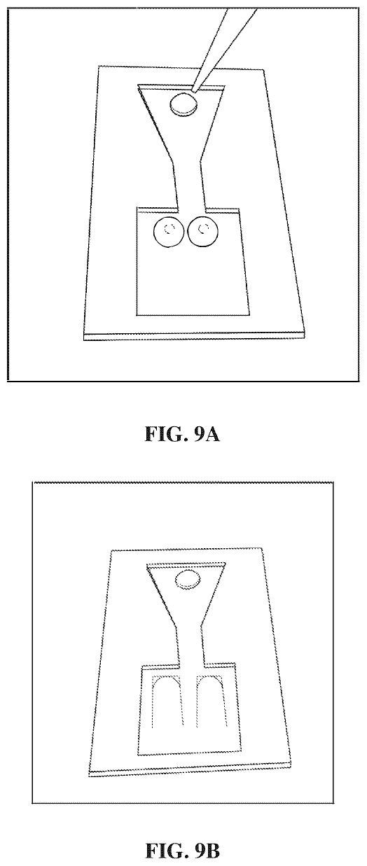

To illustrate the use of the inventive lyophilized reagent format in a microfluidic device, LyoDots (see Example 2, above) containing different dyes were prepared and then incorporated into an acrylic microfluidic chamber with dimensions of 0.5×0.5×0.030 inches (FIG. 9A). LyoDots were adhered to the surface of the chamber by placing adhesive on the underside of the surface-modified substrate used to prepare the LyoDots so as to prevent their movement when placed in the microfluidic device. After water was introduced to the device, the LyoDot components readily dissolved and remained on their respective sides of the chamber (FIG. 9B), demonstrating spatial control of the dissolved lyophilized material. This experiment demonstrates that LyoDots can be used in devices with shallow chambers where efficient storage, dissolution, and transfer of reagents are needed. As is known, such microfluidics devices include those used to perform immunoassays and molecular assays.

### Example 4

### Lyodot™ Handling

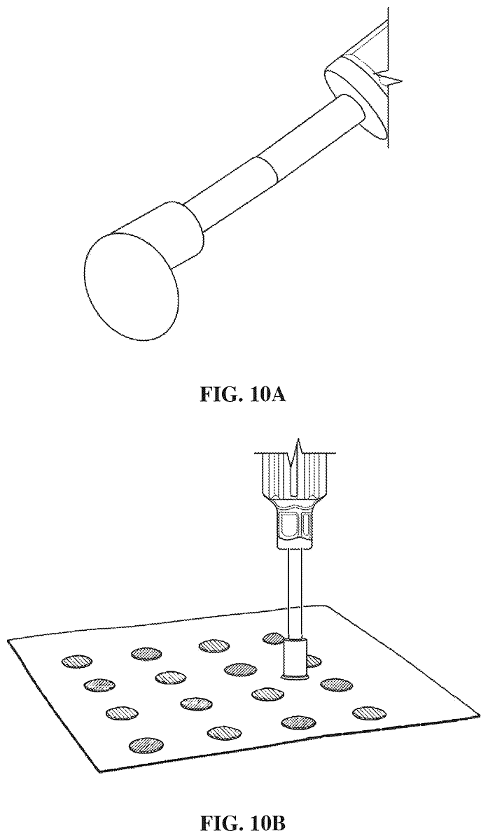

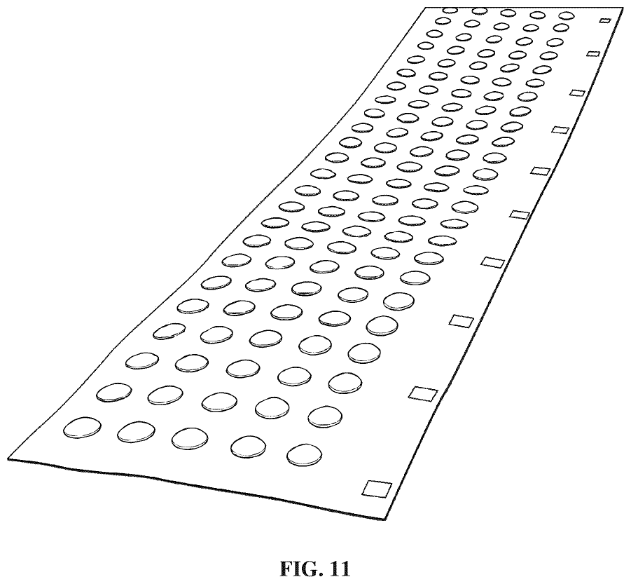

An important manufacturing consideration is robustness in handling, as occurs, for example, during the manufacture of microfluidic devices. This Example demonstrates that the lyophilized reagent format of the invention is compatible with conventional “pick and place” handling techniques that use “air tweezers” to move and manipulate components of microfluidic diagnostic devices during the assembly of such devices (see, e.g., FIG. 10A). For adaptability with high-throughput manufacturing methods, the lyophilized reagent format of the invention (e.g., LyoDots) can also be configured into sheets (FIG. 10B and FIG. 11).

Unless the context clearly requires otherwise, throughout the description above and the appended claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list, and any combination of the items in the list.

Although the invention has been described with reference to the above examples, it will be understood that modifications and variations are encompassed within the spirit and scope of the invention. Accordingly, the invention is limited only by the following claims.

What is claimed is:

1. A lyophilized reagent:substrate complex, comprising:
a solid phase substrate comprising a hydrophilicity-enhanced surface having a reagent deposit zone, wherein the substrate is a flexible membrane, film, or mesh; and
a lyophilized reagent disposed on the reagent deposit zone of the hydrophilicity-enhanced surface, wherein a maximum height of the lyophilized reagent is less than an outer diameter of the lyophilized reagent.

2. The complex according to claim 1, further comprising an adhesive layer disposed on a surface of the solid phase substrate that is opposite the substrate's hydrophilicity-enhanced surface.

3. The complex according to claim 1, wherein the lyophilized reagent comprises a convex or concave upper surface and a substantially flat bottom surface.

4. The complex according to claim 1, wherein the lyophilized reagent comprises a height that is reduced as compared to what the height of the lyophilized reagent would have been if the surface of the solid phase substrate was not a hydrophilicity-enhanced surface.

5. The complex according to claim 1, wherein the complex is defined by a maximum height and an outer diameter, wherein the maximum height is equal to or less than about 50% of the outer diameter.

6. The complex according to claim 1, wherein the complex has a disc shape.

7. The complex according to claim 1, wherein the lyophilized reagent comprises a moisture content of less than about 10% by weight.

8. The complex according to claim 1, wherein the lyophilized reagent is formed from a volume of liquid reagent of about 5000 μl or less prior to lyophilization.

9. The complex according to claim 8, wherein at least the reagent deposit zone of the hydrophilicity-enhanced surface of the substrate is treated to stabilize the lyophilized reagent.

10. A substrate that comprises a plurality of lyophilized reagent:substrate complexes according to claim 1.

11. The substrate according to claim 10, wherein some or all of the plurality of lyophilized reagent:substrate complexes comprise the same or different dried reagents.

12. The complex according to claim 8, further comprising a second lyophilized reagent layered on top of the lyophilized reagent disposed on the reagent deposit zone, wherein the second lyophilized reagent comprises a formulation that differs from that of the lyophilized reagent disposed on the reagent deposit zone of the hydrophilicity-enhanced surface of the substrate.

13. The complex according to claim 12, further comprising at least one more lyophilized reagent layer on top of the second lyophilized reagent, wherein the at least one more lyophilized reagent layer has a different reagent formulation from the first and second lyophilized reagents.

14. An assay device comprising the complex according to claim 1, wherein the assay device is optionally a microfluidic device.

15. The complex according to claim 1, wherein the reagent deposit zone has a periphery that is bounded by a perimeter region of the substrate, wherein the perimeter region comprises one or more recessed portions that extend partially or entirely through the substrate so that the lyophilized reagent:substrate complex can be detached from the substrate.

16. (canceled)

17. (canceled)

18. A method of performing an assay to detect a target analyte in a test sample, comprising:
a) contacting a lyophilized reagent:substrate complex according to claim 1 with a test sample to produce a reaction mixture; and
b) if the target analyte is present in the test sample, detecting the target analyte in the reaction mixture.

19. The complex according to claim 1, wherein the flexible membrane or mesh is a polymer flexible membrane or mesh.

20. The complex according to claim 19, wherein the polymer flexible membrane or mesh is water soluble.

21. The complex according to claim 15, wherein the one or more recessed portions extend at least about 50% or more through the substrate.

22. The assay device according to claim 14, wherein the microfluidic device is a lateral flow diagnostic device.
